(19) United States
(12) Patent Application Publication
(10) Pub. No.: US 2006/0246591 A1
(43) Pub. Date: Nov. 2, 2006

(54) Method and device for controlling at least one operating parameter of an electrolytic bath

(76) Inventors: Harald Engler, Delmenhorst (DE); Jochen Heinzinger, Vierkirdnen (DE); Konrad Jung, Doetlingen (DE); Klaus D. Rayhjen, Bremen (DE); Matthias Schmidt, Brehna (DE)

Correspondence Address: KENYON & KENYON LLP, ONE BROADWAY, NEW YORK, NY, 10004, US

(21) Appl. No.: 10/551,478

(22) PCT Filed: Mar. 29, 2004
(86) PCT No.: PCT/DE04/00653

§ 371 Date: Jul. 6, 2006

(30) Foreign Application Priority Data
Mar. 29, 2003 (DE) 103 14 279.7

(51) Int. Cl. G01N 33/00

(52) U.S. Cl. 436/55

(57) ABSTRACT

A method and a device for controlling at least one operating parameter of an electrolytic bath may provide for the production of improved quality platings, in which the use of chemicals may be reduced. The concentration of at least one bath component is determined, and the concentration values are processed in a control device in order to obtain correcting variables of a control element, in which the operating parameter is changed in line with setpoint values. The concentration is determined by extracting a sample from the bath. The sample is excited by electromagnetic radiation, and the spectrum of the light emitted by the sample is analyzed. The device includes an installation for transferring at least one sample of a bath to an array for determining the concentration. The array for determining the concentration of at least one bath component includes a laser directed at the sample, and the array for determining the concentration includes an array for spectral analysis of the light emitted by the sample.

### FIELD OF THE INVENTION

The present invention relates to a method for controlling at least one operating variable of an electrolytic bath and to a device for implementing the method.

### BACKGROUND INFORMATION

Galvanic methods are used for manufacturing workpieces having coatings for corrosion prevention, for decorative purposes and for preparations for paint work. An electroplating plant is made up of a series of process baths, in each of which an electrolytic plating process takes place, and of at least two rinse baths per process bath, at least one rinse bath normally being operated as a circulation rinse bath. The operating variables of the processes proceeding in the baths must be controlled and/or regulated as a function of different parameters. If processes are controlled manually by an operator according to the operator's store of experience, this will result in strong fluctuations of the process conditions, which leads to changing qualities of the electroplated products and to a high consumption of process substances and process adjuvants.

In the manual operation of an electroplating plant, the bath compositions are monitored by concentration measurements in samples, a manual withdrawal of the sample and an external analysis of the sample being time-consuming and cost-intensive and not allowing for a simultaneous control of the electroplating operation. The composition of the process baths directly affects the quality of the coatings. If the concentrations of the active substances such as chromium, nickel or zinc, for example, are too low in the process baths, then the metals will be deposited incompletely and in insufficiently thick layers. Furthermore, the process baths accumulate foreign ions through carryovers from previous baths and as a consequence of chemical reactions of the workpieces with foreign ions. This causes the layers to be deposited to become inhomogeneous and the inorganic corrosion protection or the preparation for the organic corrosion protection to be insufficient.

The rinse baths are used to rinse off the previously electroplated workpieces. This is to avoid carryovers with foreign ions into the subsequent process steps or into the environment. Rinse baths are recovered using ion exchangers or a reverse osmosis. Their preparation results in substantial quantities of waste water and sludges. The chemicals and waters withdrawn must again be fed into the electroplating plant as educts, which is uneconomical and ecologically dubious. This particularly applies when the rinse baths for reasons of producing a sufficient rinse effect are operated as circulatory rinse baths at a constant high volume flow. It is conventional to control the rinse bath composition according to the degree of contamination. Manually withdrawing samples from the rinse baths may result in the disadvantages described with respect to the process baths.

German Published Patent Application No. 197 36 350 describes a method for regulating the concentration of substances in electrolytes in which the content of oxidized redox ions in an electrolytic auxiliary cell is lowered to the precise extent to which metal is dissolved by the entered oxygen in the electrolyte. The regulation of the metal content occurs via the adjustable current of an auxiliary cell such that the overall electrolytic system is in equilibrium. Signals of an analyzer for determining the metal ion content of the metal to be deposited in the electroplating plant can be supplied to the current controller of the auxiliary cell.

The method for the electrolytic deposition of metals from electrolytes described in German Published Patent Application No. 44 05 741 uses additives of organic process components to achieve certain physical properties. To avoid complex concentration measurements, the organic additives are continuously added. An injection point in the pipeline system of an electrolyte circuit near an electrolytic cell may be chosen as the dosing point. The use of porous partition walls ensures that the organic process compounds are located only in the cathode space which is free of the aggressive oxidized stage of the redox agent.

In the method for dosing process baths described in German Published Patent Application No. 196 00 857, concentrates are added to the baths for replenishing the ongoing consumption of chemicals. For this purpose, a portion of a used bath solution is continuously withdrawn, while at the same time fresh bath solution is added in the same quantity. A measurement of concentration is unnecessary.

German Published Patent Application No. 197 27 939 describes a method for dosing rinsing fluid in which the weight of a carried-over quantity of solution is determined by weight measurements of the object to be electroplated and if necessary of the support of the object. From the measured carry-over values it is possible to calculate the resulting concentration changes in the respective bath stations. This is done by a data processing system to which an open-loop or closed-loop control circuit for the dosing of rinsing water is connected such that slightly changed solution concentrations can be automatically adjusted to a setpoint value.

Furthermore, it is described in PCT International Published Patent Application No. WO 00/00811 to use the methods of laser Raman spectroscopy for analyzing pharmaceutical substances.

Japanese Published Patent Application No. 11-118796 describes a system for analyzing proteins in urine, in which a urine sample is mixed with a diluent and a dyeing agent and is supplied via pipelines to an optical scattered light analyzer. In the scattered light analyzer, the sample fluid is exposed to laser light. The composition of the scattered light changes as a function of the protein content of the sample fluid. The analysis time and the quantity of the sample fluid are changed as a function of the absorption of the measured light.

In the system for optical substance analysis described in European Published Patent Application No. 1 059 708, a substance sample is exposed to a bundled laser light. The substance interacts with the laser light.

The foregoing methods of substance analysis are concerned with the investigation of individual samples which are prepared at the site of the analysis system and are supplied to the analysis system. With respect to the handling of the samples and with respect to the interaction with open-loop and closed-loop control devices, the analysis systems are not designed for the process control of electroplating plants.

### SUMMARY

Example embodiments of the present invention may provide a method and a device for controlling at least one operating variable of an electrolytic bath, which may allow for the production of coatings of improved quality, while reducing the use of chemicals.

The method for controlling at least one operating variable of an electrolytic bath is based on the measurement of concentration of a bath component using an electromagnetic radiation which excites the sample taken from the bath such that light is emitted. The concentration may be ascertained from the spectrum of the emitted light. Using the measured concentration values, it is possible to control or regulate various operating variables. In this context, an operating variable is understood in the broadest sense as any physical variable, the change of which influences the quality of a coating and the quantity of the chemicals used. Important operating variables may include, for example, the composition of the bath, the bath temperature, the movement of the electrolyte and of the objects to be galvanized, the degree of contamination or the electric current in a bath, etc.

Example embodiments of the present invention may be used to control or regulate an electroplating plant with the aid of a process-integrated analysis, the analysis being, e.g., based on the laser-induced emission spectral analysis. Using the laser-induced emission spectral analysis, it may be possible to control the contamination of a galvanic bath or rinse bath in an advantageous manner. A system for the laser-induced emission spectral analysis may include a laser, which is used to vaporize, by a number of laser pulses, a fluid sample withdrawn from the bath. The quantity of the fluid may be small, e.g., having a volume of less than 1 ml.

The ionogenic composition of the substances contained in the fluid is determined by a downstream spectrometer. The contactless measuring method may have no or only a slight sensitivity to contamination by foreign ions. The measuring process may allow for a rapid bath analysis without complicated sample preparation. A measuring time of less than three seconds may be achieved in practice, which represents a quasi simultaneous detection of the state of a bath. A decomposition of the sample may not be required. The time-resolved measurements of the concentrations may be made without the use of a protective gas at air atmosphere. The spectroscopic measured values are transmitted to a computer which is part of the open-loop or closed-loop control system of the electroplating plant. As the result of the execution of a measured value processing program, control outputs for control elements are generated which control, e.g., the re-sharpening or discharging of a process bath when specified concentrations of process bath or interfering substances are undershot or exceeded. This also provides for the possibility of controlling the regeneration of the process bath, e.g. using diffusion analysis in acidic scouring or diaphragm electrolysis in chromium-containing process solutions, via concentration measurements of interfering components. In addition to optimizing the electroplating operation, this may be used for a process-integrated quality assurance since the process bath quality directly influences the result of electroplating.

If the ionogenic contaminations in the rinse baths are measured, then this allows for a control of the rinse bath regeneration, i.e., of the volume flow to be regenerated. This may allow for the use of energy, process adjuvants such as coagulants and precipitants, and of water or waste water to be minimized.

In order to prevent the intermixing of the individual process baths and thus a falsification of the measured values, samples from each bath are separately supplied to the spectrometer via a decentralized pipeline system. With regard to a good mixture, high flow velocities of the sample fluids may be achieved in the pipes of the pipeline system. At low sample volumes, the pipeline system may be provided in a cost-effective manner having small flow cross-sections. The separate pipeline system may save time-consuming and costly in-between cleanings of the pipelines using distilled water and subsequent drying using compressed air, which may be necessary if all baths were connected to the spectrometer via only one sample feed.

In an example embodiment of the device for controlling at least one operating variable of an electrolytic bath, the pipeline system ends at a sample plate, it being possible for the sample fluid from a particular bath to be squirted automatically and in a clocked manner onto the sample plate. A sample plate may take the form of a carousel for individual samples, laser light acting directly onto a sample on the sample plate. Following the analysis of the light emanating from the sample, the sample is removed from the sample plate using suitable cleaning devices and the sample plate is provided with a fresh sample. The sample plate may be provided with samples of only one bath as well as samples from different baths.

The spectroscopic measured values obtained may be transmitted directly to a master computer of the electroplating plant for calculating control outputs. For a process-integrated quality assurance, the measured data may be stored for archiving the bath states, e.g., the process bath states. There is the possibility of recording the time-dependency of the concentrations and the detection of concentration intervals. The method and the device may allow for the process-integrated detection, e.g., of aluminum, copper, cadmium, chromium, iron, zinc, etc., as well as of other elements in process and/or rinse baths. It is possible to ascertain concentrations of the mentioned substances in the process baths in the range of, e.g., 1 to 100 g/l and in the rinse baths in the range below, e.g., 100 ppm.

Example embodiments of the present invention are explained below with reference to the appended Figure.

### BRIEF DESCRIPTION OF THE DRAWING

FIG. 1 is a schematic view of an electroplating plant for implementing a method according to an example embodiment of the present invention.

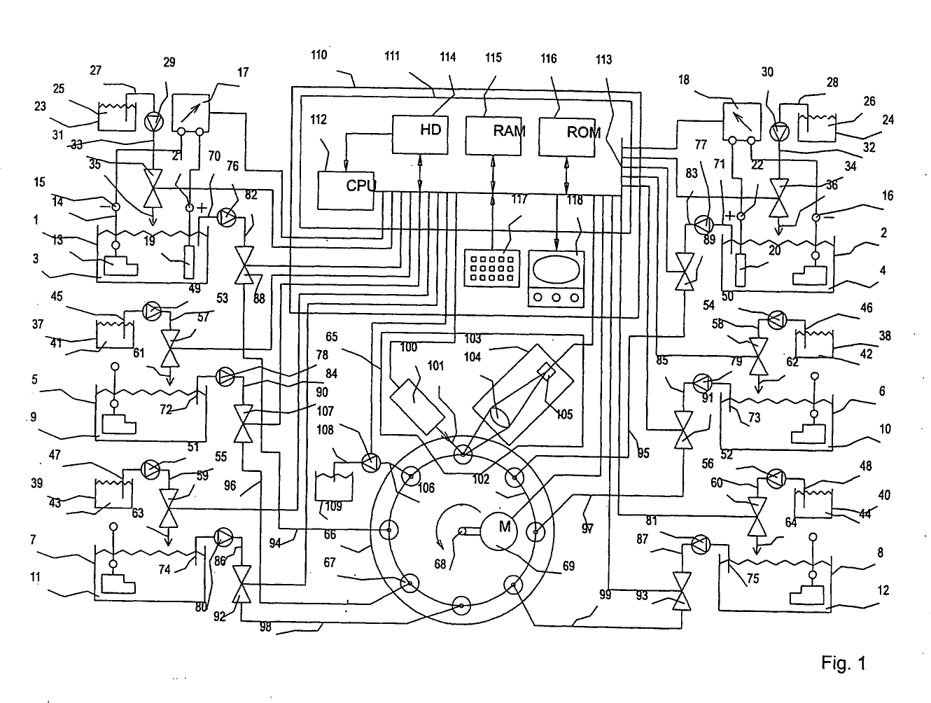

### DETAILED DESCRIPTION

The electroplating plant includes two containers 1, 2 for process baths 3, 4 and of four containers 5 to 8 for rinse baths 9 to 12, which in the process sequence are respectively downstream of process baths 3, 4. Salt of a coating metal is dissolved in each of process baths 3, 4. Workpieces 13 to be coated are suspended from carrier devices 14, which are connected to conveyer devices. Workpieces 13 are completely immersed in a process bath 3, 4 or rinse bath 9 to 12. Via carrier device 14, workpieces 13 in process bath 3, 4 are in each case connected to the negative pole 15, 16 of a controllable power source 17, 18. Process baths 3, 4 moreover include electrodes 19, 20 leading to the positive pole 21, 22 of the respective power source 17, 18. When current flows through process baths 3, 4, atoms of the coating metal are deposited by a chemical reduction onto previously thoroughly cleaned workpieces 13. To maintain the equilibrium between the atoms deposited on workpieces 13 and the atoms contained in salt solutions of the coating metal, a dosing device for the salt is located on each process bath 3, 4. A dosing device includes a reservoir 23, 24 for a highly concentrated saline solution 25, 26, an intake line 27, 28, a dosing pump 29, 30, a connecting line 31, 32, a controllable dosing valve 33, 34 and an outlet 35, 36. For regenerating rinse baths 9 to 12, dosing devices are provided, which for each rinse bath 9 to 12 include a reservoir 37 to 40 for a non-contaminated rinse bath fluid 41, 44, an intake line 45 to 48, a dosing pump 49 to 52, a connecting line 53 to 56, a dosing valve 57 to 60 and an outlet 61 to 64.

For monitoring the concentration of the coating metals in process baths 3, 4 and the degree of contamination by foreign ions in process baths 3, 4 and in rinse baths 9 to 12, a device 65 for laser-induced spectral analysis is provided. Device 65 includes a sample plate 66 having concentrically arranged sample hollows 67. Sample plate 66 is arranged such that it is able to rotate about its center axis 68 with the aid of a stepping motor 69. Device 65 is connected via a pipeline system to process baths 3, 4 and rinse baths 9 to 12. For each process bath 3, 4 or rinse bath 9 to 12, the pipeline system includes an intake line 70 to 75, a pump 76 to 81, a connecting line 82 to 87, a controllable valve 88 to 93 and outlet lines 94 to 99, which in each case lead to a sample hollow 67. Device 65 furthermore includes a laser 100, the beam 101 of which points to the reference circle 102 of sample hollows 67 on sample plate 66. Device 65 furthermore includes a spectroscope 103 having an imaging system 104 and a radiation receptor 105. A cleaning device for sample hollows 67 is associated with device 65. The cleaning device includes suction line 106 beginning at reference circle 102, a suction pump 107 and a line 108 leading to a waste container 109.

For controlling and regulating the composition of process baths 3, 4 and rinse baths 9 to 12, an open-loop and closed-loop control device 110 is provided which may be part of a control station of the electroplating plant. In addition to display and operating devices, power supply devices, signal converter and processing systems and control elements, open-loop and closed-loop control device 110 includes a computer 111. Computer 111 includes a central processor 112, which is connected via a bus system 113 to a hard disk drive 114, a random access memory 115 and a read-only memory 116. A keyboard 117 and a screen 118 are connected to bus system 113. Central processor 112 is used for the temporal coordination and control of all elements connected to bus system 113. Random access memory 115 is used for storing temporary instructions or data. Read-only memory 116 includes invariable instructions, data and programs, which are required for the correct functioning of computer 111. Hard disk drive 114 is a memory having a large capacity for storing programs and data for the implementation of the processing of measured values and the calculation of control variables. Keyboard 117 allows for the input of data on the part of an operator. Screen 118 is used to issue data and instructions to the operator. Via measurement and control lines 119 represented as dashed lines and using suitable interfaces, dosing valves 33, 34, 57 to 60, valves 88 to 93, power sources 17, 18, suction pump 107, stepping motor 69, laser 100 and spectroscope 103 are connected to bus system 113.

Using the electroplating plant described above, the method may be implemented as follows:

With the aid of carrier devices 14 and the conveyer devices, workpieces 13 are transported in succession through process bath 3, rinse baths 9, 11, process bath 4 and rinse baths 10, 12. In the passage through process baths 3, 4, the galvanic salt solutions deplete of ions of the coating metal. In the passage through rinse baths 9 to 12, the rinse solutions are contaminated with foreign ions. The method is based on continuously measuring the concentrations of the coating metal ions in process baths 3, 4 and the degrees of contamination of process baths 3, 4 and of rinse baths 9 to 12 with foreign ions using device 65. For this purpose, small quantities of process baths 3, 4 and rinse baths 9 to 12 are in each case transported by pumps 76 to 81 through connecting lines 82 to 87, valves 88 to 93 and outlet lines 94 to 99 into one of sample hollows 67. For this purpose, open-loop and closed-loop control device 110 briefly opens one of valves 88-93 such that, in accordance with the program specified by computer 111, samples of one or several process baths 3, 4 or rinse baths 9 to 12 are provided on sample plate 66 for measuring using device 65. Stepping motor 69 causes sample plate 66 to rotate about center axis 68 such that a sample hollow 67 together with a sample is brought into the direction of beam 101 or the optical axis of the path of the measuring beam of spectroscope 103. Laser beam 101 excites the sample in a short time such that the sample emits light, which for spectral analysis strikes through optical system 104 onto radiation receptor 105. The spectrum of the light on radiation receptor 105 is characteristic for the elements contained in a sample and their concentrations. The measured values of spectroscope 103 are supplied to computer 111 where they are processed.

If the concentrations of the coating metal ions in process baths 3, 4 fall below specified threshold values, then control commands are output to dosing valves 33, 34 via bus system 113 such that dosing valves 33, 34 open for specified time spans. While a dosing valve 33, 34 opens, concentrated saline solution 25, 26 is supplied by dosing pumps 29, 30 to the respective process bath 3, 4 for refreshment. Concentrated saline solution 25, 26 intermixes with the depleted process bath solution such that the concentration of the coating metal ions is essentially kept constant aside from small deviations.

The time characteristic of the growth of a coating on workpieces 13 may be influenced by adjusting the current flow in the process baths. For this purpose, control signals are sent from open-loop and closed-loop control device 110 to power sources 17, 18.

If the concentration of the foreign ions in rinse baths 9 to 12 exceed specified threshold values, then opening instructions are sent from open-loop and closed-loop control device 110 to dosing valves 57 to 60. As dosing valves 57 to 60 are opened for a specified duration, fresh rinse bath solution is supplied to the respective rinse bath 9 to 12, while used rinse bath solution is removed.

Following the analysis of a sample in a sample hollow 67, sample plate 66 is rotated further by stepping motor 69 such that the respective sample hollow 67 reaches the location of suction line 106. Via bus system 113, suction pump 107 receives an activation signal which puts suction pump 107 into operation and thus removes the rest of the sample from sample hollow 67 into waste container 109.

A device hereof is not limited to the exemplary embodiment illustrated. The equipment of the pipeline system with pumps 76 to 81 and valves 88 to 93 is only exemplary. Instead of sample plate 66, device 65 may be equipped with other devices for handling samples. In place of suction pump 107 and suction line 106, other suitable cleaning devices for sample containers may be provided. The device for refreshing process baths 3, 4 and for regenerating rinse baths 9 to 12 may be designed differently than described. Furthermore, the number of process baths 3, 4 and rinse baths 9 to 12 may be adapted to the prevailing requirements.

### LIST OF REFERENCE SYMBOLS USED

1, 2 container 68 center axis
3, 4 process bath 69 stepping motor
5-8 container 70-75 intake line
9-12 rinse bath 76-81 pump
13 workpiece 82-87 connecting line
14 carrier device 88-93 valve
15, 16 negative pole 94-99 outlet line
17, 18 power source 100 laser
19, 20 electrode 101 beam
21, 22 positive pole 102 divided circle
23, 24 reservoir 103 spectroscope
25, 26 saline solution 104 imaging system
27, 28 intake line 105 radiation receptor
29, 30 dosing pump 106 suction line
31, 32 connecting line 107 suction pump
33, 34 dosing valve 108 line
35, 36 outlet 109 waste container
37-40 reservoir 110 open-loop and closed-loop-control device
41-44 rinse bath fluid 111 computer
45-48 intake line 112 central processor
49-52 dosing pump 113 bus system
53-56 connecting line 114 hard disk drive
57-60 dosing valve 115 access memory
61-64 outlet 116 read-only memory
65 device 117 keyboard
66 sample plate 118 screen
67 sample hollow

1-10. (canceled)

11. A method for controlling at least one operating variable of an electrolytic bath, comprising:
ascertaining a concentration of at least one bath component;
processing concentration values in a control device into control variables of a control element; and
changing the operating variable by the control element in accordance with setpoint inputs;
wherein the concentration is ascertained in the ascertaining step by withdrawing a sample from the bath, exciting the sample by electromagnetic radiation and analyzing a spectrum of light emitted by the sample.

12. The method according to claim 11, further comprising supplying the sample via a line to at least one sample container.

13. The method according to claim 12, wherein the supplying step includes successively filling a plurality of sample containers with the sample, the method further comprising carrying the sample containers past a spectroscopic measurement device.

14. The method according to claim 11, further comprising supplying several samples onto a sample plate and carrying the samples past a spectroscopic measurement device by rotating the sample plate.

15. The method according to claim 11, wherein the sample is excited optically.

16. The method according to claim 11, wherein the sample is excited optically by a laser beam.

17. A device for controlling at least one operating variable of at least one electrolytic bath, comprising:
an arrangement adapted to ascertain a concentration of at least one bath component;
a setpoint adjustment device for the operating variable;
an open-loop and closed-loop control device connected to the arrangement and to the setpoint adjustment device, the control device including a control element adapted to change the operating variable; and
a device adapted to transmit at least one sample of the bath to the arrangement;
wherein the arrangement includes a laser directed onto the sample and a spectral analysis device adapted for spectral analysis of light emitted by the sample.

18. The device according to claim 17, wherein the device adapted to transmit the at least one sample includes a pipeline system in an electroplating plant having several baths.

19. The device according to claim 17, further comprising at least one pipe having an intake end immersed into a corresponding one of the baths and an outlet end arranged at a rotatable sample plate that is partially located in a radiation range of the laser.

20. The device according to claim 19, further comprising a device adapted to remove analyzed samples from the sample plate.

21. A device for controlling at least one operating variable of at lease one electrolytic bath, comprising:
means for ascertaining a concentration of at least one bath component;
means for adjusting a setpoint of the operating variable;
open-loop and closed-loop control means connected to the ascertaining means and the adjusting means;
means for changing the operating variable; and
means for transmitting at least one sample of the bath to the ascertaining means;
wherein the ascertaining means includes laser means directed onto the sample and means for spectral analysis of light emitted by the sample.
